(19) United States
(12) Patent Application Publication
(10) Pub. No.: US 2022/0331327 A1
(43) Pub. Date: Oct. 20, 2022

(54) THE USE OF PARAXANTHINE TO IMPROVE PERFORMANCE IN VIDEO GAMERS

(71) Applicant: Ingenious Ingredients, LP, Lewisville, TX (US)

(72) Inventors: Ralf Jäger, Whitefish Bay, WI (US); Martin Purpura, Spring, TX (US); Shawn D. Wells, Frisco, TX (US); Kylin Liao, Plano, TX (US)

(21) Appl. No.: 17/710,787

(22) Filed: Mar. 31, 2022

Related U.S. Application Data
(60) Provisional application No. 63/168,458, filed on Mar. 31, 2021.

(51) Int. Cl. A61K 31/522; A61K 31/593; A61K 31/122; A61K 31/405; A61K 33/04; A61K 31/198; A61K 31/047; A61K 31/07; A61K 36/07; A61K 36/35; A61K 36/81; A61P 25/28

(52) U.S. Cl. CPC A61K 31/522; A61K 31/593; A61K 31/122; A61K 31/405; A61K 33/04; A61K 31/198; A61K 31/047; A61K 31/07; A61K 36/07; A61K 36/35; A61K 36/81; A61P 25/28; A63F 13/85

(57) ABSTRACT

Disclosed herein is a method for increasing video game performance in a subject by administering to the subject with a composition comprising from about 2 mg to about 800 mg of paraxanthine. In certain embodiments, paraxanthine is present in the composition in amount from about 20 mg to about 600 mg. Further disclosed herein is a method for enhancing mood of a subject during electronic gameplay or preventing decrease in mood of a subject during electronic game play comprising administering to the subject a composition comprising from about 2 mg to about 800 mg of paraxanthine. In certain embodiments, administration of the composition to the subject decreases, or prevents the increase of, one or more of tension, depression, anger, fatigue and/or confusion in the subject during electronic game play relative to a subject who has not been administered the composition.

### CROSS-REFERENCE TO RELATED APPLICATION(S)

This application claims priority to U.S. Provisional Application No. 63/168,458 filed Mar. 31, 2021 and entitled “THE USE OF PARAXANTHINE TO IMPROVE PERFORMANCE IN VIDEO GAMERS,” which is hereby incorporated by reference in its entirety under 35 U.S.C. § 119(e).

### TECHNICAL FIELD

The disclosed technology relates generally to compositions, methods, and system for utilizing paraxanthine alone and in combination for use in increasing video game performance in a subject. More particularly, the disclosure relates to paraxanthine and other compounds, whether produced synthetically or derived from natural sources, and use of these chemical compounds to provide physiological benefits, which may vary according to paraxanthine concentration and the presence of synergists and antagonists.

### BACKGROUND

Competitive sporting competitions in large venues with sellout crowds are no longer just the domain of soccer, basketball, handball, American football, or baseball. They now include competitive eSports. The term eSports is also known or referred to as, esports, e-sports, competitive (video) gaming, professional (video) gaming, or pro gaming) (each used interchangeably herein). In 2020, 1.5 billion people played video games, and 495 million people watched eSports events worldwide, with 272 million being occasional viewers, and 223 million are frequent viewers/enthusiasts. 62% of internet users watching eSports championships worldwide in 2019 are between 16-34 years old, and the global eSports audience consisted of 78% men and 22% women. In the U.S., in 2020 14% of the population identified themselves as casual fan, 5% as avid fan, and 81% are not a fan at all, indicating a tremendous growth potential.

For gamers to perform at an optimum, numerous acute and chronic aspects of performance must be addressed. Chronic protection of brain health, eye health, or joint health is needed. In order to optimize performance, numerous aspects of physical and mental needs must be addressed simultaneously. Reaction time, concentration, accuracy, attention, memory, energy, hand-eye coordination.

An arginine-based ingredient has been clinically validated to increase accuracy, decision making, and reaction time during gaming through increasing blood flow to the brain (Nutrients 2019 Oct. 1; 11(10):2326. doi: 10.3390/nu11102326.). Commercial product target at eGamers include XP Sports' Gummies for mental clarity and eye health, or XP Sports Boost, pre-game energy and focus amplifier containing L-Theanine, Ginseng, Choline, alpha-GPC, Phosphatidylserine, lutein or caffeine. While caffeine has been shown to increase attention and perceived energy, it also increases anxiety, and is potentially decremental to eSports performance.

There is a need in the art for a safe and effective ingredient (or combination of ingredients) to boost energy and confidence while increasing performance.

### BRIEF SUMMARY

Disclosed herein is a method for increasing video game performance in a subject by administering to the subject with a composition comprising from about 2 mg to about 800 mg of paraxanthine. In certain embodiments, paraxanthine is present in the composition in amount from about 20 mg to about 600 mg. In further embodiments, paraxanthine is present in the composition in amount from about 50 mg to about 400 mg. In still further embodiments, video game performance is assessed by a measure selected from: accuracy, decision making, reaction time, hand-eye coordination, or game performance. In exemplary implementations game performance in the subject is increased from about 10% to about 70% following administration of the composition.

According to certain embodiments, the composition further comprises one or more additional active agents. In certain implementations, the one or more active agents are selected from: omega-3 fatty acids, vitamin D, vitamin B, protein, selenium, fast digestive carbohydrates, vitamin K, calcium, vitamin A, ashwagandha (Withania somnifera), Acetyl L-Carnitine, tyrosine, N-acetyl-L-tyrosine, Ergothioneine, tryptophan, 5-HTP, arginine, citrulline, norvaline, GABA, Dopa (Velvet Bean), Kanna (serotonin), L-theanine, phosphatidylcholine, alpha-GPC (L-alpha glycerylphosphorylcholine), Citicoline (Cytidine diphosphate choline (CPD Choline)), Choline Bitartrate, bacopa Monnieri, Phosphatidylserine, pilocarpine, cevimeline, Amburana cearensis, Lippia sidoides, Paullinia cupana, Plathymiscium floribundum, curcuminoids, caffeine, creatine, green coffee berry, lions mane, niacin, kanna extract, saffron, SAMe, acetyl L-carnitine, huperzine A, lutein/zeaxanthin, rhodiola, theacrine, methylcobalamin, macuna methyliberine and Solanum asperum.

In certain embodiments, the composition is administered prior to the onset of play. In further embodiments, the composition is administered during video game play. In yet further embodiments, the composition is administered at repeated intervals during video game play. In still further embodiments, the composition is administered to the subject daily for a period of between about 1 and about 9 weeks.

In certain embodiments, the paraxanthine is synthetic. In further embodiments, paraxanthine is derived from a natural source.

Further disclosed herein is a method for enhancing electronic gameplay endurance in a subject comprising: providing the subject with a composition comprising from about 2 mg to about 800 mg of paraxanthine. In certain embodiments, paraxanthine is present in the composition in amount from about 50 mg to about 400 mg. In certain implementations, the subject has engaged in electronic game play for at least about 100 minutes.

Further disclosed herein is a method for enhancing mood of a subject during electronic gameplay or preventing decrease in mood of a subject during electronic game play comprising administering to the subject a composition comprising from about 2 mg to about 800 mg of paraxanthine. In certain embodiments, administration of the composition to the subject decreases, or prevents the increase of, one or more of tension, depression, anger, fatigue and/or confusion in the subject during electronic game play relative to a subject who has not been administered the composition. In certain implementations, administration of the composition to the subject decreases, or prevents the increase of, tension and/or anxiety in the subject during electronic game play relative to a subject who has not been administered the composition. In further implementations, administration of the composition to the subject increases, or prevents the decrease of, vigor in the subject during electronic game play relative to a subject who has not been administered the composition.

While multiple embodiments are disclosed, still other embodiments of the disclosure will become apparent to those skilled in the art from the following detailed description, which shows and describes illustrative embodiments of the disclosed compositions, systems and methods. As will be realized, the disclosed compositions, systems and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF DRAWINGS

FIG. 1 shows STROOP test data, according to certain embodiments.

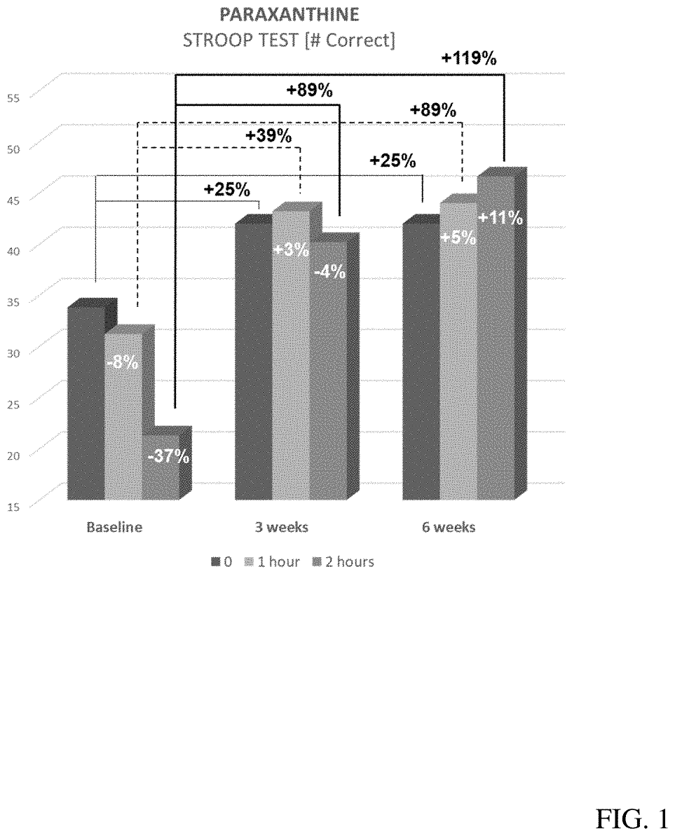

### DETAILED DESCRIPTION

Before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “subject” refers to the target of administration, e.g., an animal capable of playing video games. The term does not denote a particular age or sex. Thus, adult and child subjects, whether male or female, are intended to be covered. In one aspect, the subject is a mammal.

As used herein, the term “synergistic effect” or grammatical variations thereof means and includes a cooperative action encountered in a combination of two or more active compounds in which the combined activity of the two or more active compounds exceeds the sum of the activity of each active compound alone. The term “synergistically effective amount,” as used herein, means and includes an amount of two or more active compounds that provides a synergistic effect defined above.

As used herein, the terms “effective amount” and “amount effective” refer to an amount that is sufficient to achieve the desired result or to have an effect on an undesired condition. The specific effective dose level for any particular subject will depend upon a variety of factors including the; the specific composition employed; the age, body weight, general health, sex and diet of the subject; the time of administration; the route of administration; the rate of excretion of the specific compound employed; the duration of the treatment; drugs used in combination or coincidental with the specific compound employed and like factors well known in the art. For example, it is well within the skill of the art to start doses of a compound at levels lower than those required to achieve the desired effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual subject in the event of an adverse effects. Dosage can vary, and can be administered in one or more dose administrations daily, for one or several days. Guidance can be found in the literature for appropriate dosages for given classes of nutraceutical products.

This disclosure relates to the use chemical compositions comprising paraxanthine, either naturally or synthetically produced. In certain embodiments, the composition comprises other chemicals, including paraxanthine congeners or analogs, to provide a plurality of desirable effects. Paraxanthine analogs may include, but are not limited to, caffeine, methyl caffeine, theobromine, theophylline, liberine and methylliberine, and their variants. In certain embodiments, the disclosed composition includes one or more additional active agents (e.g., agent that additively or synergistically with paraxanthine increase video game performance). In exemplary implementations, such additional actives are chosen from: omega-3 fatty acids, vitamin D, vitamin B, protein, selenium, fast digestive carbohydrates like sugar, vitamin K, calcium, vitamin A, ashwagandha (Withania somnifera), Acetyl L-Carnitine, tyrosine, N-acetyl-L-tyrosine, Ergothioneine, tryptophan, 5-HTP, arginine, citrulline, norvaline, GAB A, Dopa (Velvet Bean), Kanna (serotonin), L-theanine, phosphatidylcholine, alpha-GPC (L-alpha glycerylphosphorylcholine), Citicoline (Cytidine diphosphate choline (CPD Choline)), Choline Bitartrate, Bacopa Monnieri, Phosphatidylserine, pilocarpine, and cevimeline Amburana cearensis, Lippia sidoides, Paullinia cupana, Plathymiscium floribundum, curcuminoids, caffeine, creatine, green coffee berry, lions mane, niacin, kanna extract, saffron, SAMe, acetyl L-carnitine, huperzine A, lutein/zeaxanthin, rhodiola, theacrine, methylcobalamin, macuna, methyliberine, taurine, electrolytes like sodium, potassium, magnesium, and calcium, inositol enhanced arginine silicate, nitrates, adenosine triphosphate, valerian root, quercetin, hesperidin, milk thistle, and Solanum asperum.

In certain embodiments, the composition administered to the subject does not contain caffeine. In further embodiments, the subject abstains from caffeine during performance of the disclosed methods.

Further disclosed herein is a method for increasing video game performance in a subject by providing the subject with a composition comprising about 2 mg to about 800 mg of paraxanthine. Although doses will vary from subject to subject, suitable daily doses are in the range of about 1 to about 1000 mg (e.g., about 1 mg, about 5 mg, about 10 mg, about 15 mg, about 20 mg, about 25 mg, about 30 mg, about 35 mg, about 40 mg, about 45 mg, about 50 mg, about 75 mg, 100, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, or about 1000 mg, and the like, or any range or value therein) per subject, administered in single or multiple doses. In certain aspects, paraxanthine is present in the composition in amount from about 20 mg to about 600 mg. In further aspects, paraxanthine is present in the composition in amount from about 50 mg to about 400 mg. In certain aspects, paraxanthine is present in the composition in amount from about 20 mg to about 600 mg. In further aspects, paraxanthine is present in the composition in amount from about 50 mg to about 400 mg.

In certain embodiments, the disclosed compositions are nutraceutical compositions. Exemplary nutraceutical compositions of the present disclosure may be formulated or used as a standalone composition, or as a nutritional or bioactive component in food, a functional food, a beverage, a bar, a food flavor, a medical food, a dietary supplement, or a herbal product. A medium generally accepted in the art includes all pharmaceutically or nutraceutically acceptable carriers, diluents or excipients therefor.

Paraxanthine exhibits a wide variety of effects depending on dosage. The presence of other ingredients may also modulate its effects. In certain embodiments, administration of a paraxanthine containing composition to a subject increases video game performance in the subject. Assessing impact on video game performance may be accomplished by number of measures known in art. Many games provide metrics to assess performance as part of the game (e.g., point scores). In certain embodiments, video game performance is assessed by a measure selected from: accuracy, decision making, reaction time, hand-eye coordination, and game performance. In certain implementations, video game performance is assessed by a combination of the foregoing measures, each of which paraxanthine administration may serve to improve. In certain embodiments, paraxanthine increases game performance by reducing visual fatigue and/or increasing visual acuity.

In certain embodiments, administration of the composition to the subject increases subject's game performance from about 5% to about 70%. In further embodiments, the increase in the subject's game performance is from about 10% to about 60%. In yet further embodiments, the increase in the subject's game performance from about 20% to about 50%. In still further embodiments, the increase in the subject's game performance is at least about 30%. In certain embodiments, game play performance is assessed relative to the subject's level of game play performance at the beginning of play (e.g., during the first 30 minutes of play). In further embodiments, a subject's electronic game play performance is assessed relative to a subject who has played for a comparable period of time but has received placebo.

According to certain embodiments of the disclosed method, the composition is administered prior to the onset of video game play. In further embodiments, the composition is administered during video game play. In still further embodiments, the composition is administered before and during video game play. In yet further embodiments, the composition is administered and regular intervals during video games play. In certain alternative embodiments, the composition is administered as needed during video game play.

In still further embodiments, the composition is administered to the subject daily for a period of between about 1 and about 9 weeks. In further embodiments, the disclosed composition is administered daily to the subject for between about 3 and about 6 weeks. In further embodiments, the composition is administered to the subject daily for a period of at least about 5 weeks.

Further disclosed herein is a method for enhancing electronic game play endurance in a subject through administration of the instantly disclosed paraxanthine containing compositions. Prolonged game play frequently results in decreasing performance over time. As used herein “enhancing electronic game play endurance” means preventing or limiting the decline of performance over time or, in certain embodiments, slowing the rate at which performance declines over time. In certain embodiments, electronic game play endurance is assessed relative to the subject's level of game play at the beginning of play (e.g., during the first 30 minutes of play). In further embodiments, a subjects electronic game play endurance is assessed relative to a subject who has played for a comparable period of time but has received placebo or the same subject who receives placebo during a separate game play session.

Further disclosed for enhancing mood of a subject during electronic gameplay or preventing decrease in mood of a subject during electronic game play by administering to the subject a composition comprising paraxanthine. During electronic game play, certain subjects experience a decrease in mood that may adversely affect gaming performance. In certain embodiments, administration of the instantly disclosed composition enhances mood of the subject during game play. In further embodiments, administration of the instantly claimed composition prevents decrease in mood during game play. In certain embodiments, administration of the disclosed composition prevents the increase of one or more of tension, depression, anger, fatigue and/or confusion. In further embodiments, administration of the disclosed composition causes a decrease in one or more of tension, depression, anger, fatigue and/or confusion. In further embodiments, administration of the disclosed composition results in a decrease or prevents an increase of tension and/or anxiety. In yet further embodiments, administration of the composition results in an increase of vigor in the subject. In certain embodiments, changes in subject mood is assessed relative to the subject's mood at the beginning of play (e.g., during the first 30 minutes of play). In further embodiments, a subject's mood is assessed relative to a subject who has played for a comparable period of time but has received placebo.

An advantage of using the invention may be the reduced likelihood that a person develops a tolerance to chemical compositions in accordance with the principles of the invention. That is, a person may not become desensitized to the effects induced.

The administration of the disclosed compositions to a subject may include any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, intradermal administration, buccal administration, and parenteral administration, including injectable such as intravenous administration, intra-arterial administration, intramuscular administration, and subcutaneous administration.

### Nutritional Supplements

The compositions of the disclosure may take the form of dietary supplements or may themselves be used in combination with dietary supplements, also referred to herein as food supplements.

Nutritional supplements may be found in many forms such as tablets, capsules, soft gels, gel caps, liquids, or powders. Some dietary supplements can help ensure an adequate dietary intake of essential nutrients; others may help reduce risk of disease.

### Food Products

The compositions of the disclosure may take the form of a food product. Here, the term “food” is used in a broad sense and covers food and drink for humans as well as food and drink for animals (i.e. a feed). Preferably, the food product is suitable for, and designed for, human consumption.

The food may be in the form of a liquid, solid or suspension, depending on the use and/or the mode of application and/or the mode of administration.

When in the form of a food product, the composition may comprise or be used in conjunction with one or more of: a nutritionally acceptable carrier, a nutritionally acceptable diluent, a nutritionally acceptable excipient, a nutritionally acceptable adjuvant, a nutritionally active ingredient.

By way of example, the compositions of the disclosure may take the form of one of the following: A fruit juice; a beverage comprising whey protein: a health or herbal tea, a cocoa drink, a coffee drink, a yoghurt and/or a drinking yoghurt, a cheese, an ice cream, a desserts, a confectionery, a biscuit, a cake, cake mix or cake filling, a snack food, a fruit filling, a cake or doughnut icing, an instant bakery filling cream, a filling for cookies, a ready-to-use bakery filling, a reduced calorie filling, an adult nutritional beverage, an acidified soy/juice beverage, a nutritional or health bar, a beverage powder, a calcium fortified soy milk, or a calcium fortified coffee beverage.

### Food Ingredients

Compositions of the present disclosure may take the form of a food ingredient and/or feed ingredient.

As used herein the term “food ingredient” or “feed ingredient” includes a composition which is or can be added to functional foods or foodstuffs as a nutritional and/or health supplement for humans and animals.

The food ingredient may be in the form of a liquid, suspension or solid, depending on the use and/or the mode of application and/or the mode of administration.

### Functional Foods

Compositions of the disclosure may take the form of functional foods.

As used herein, the term “functional food” means food which is capable of providing not only a nutritional effect, but is also capable of delivering a further beneficial effect to the consumer.

Accordingly, functional foods are ordinary foods that have components or ingredients (such as those described herein) incorporated into them that impart to the food a specific function—e.g. medical or physiological benefit—other than a purely nutritional effect.

Although there is no legal definition of a functional food, most of the parties with an interest in this area agree that they are foods marketed as having specific health effects beyond basic nutritional effects.

Some functional foods are nutraceuticals. Here, the term “nutraceutical” means a food which is capable of providing not only a nutritional effect and/or a taste satisfaction, but is also capable of delivering a therapeutic (or other beneficial) effect to the consumer. Nutraceuticals cross the traditional dividing lines between foods and medicine.

### Medical Foods

Compositions of the present disclosure may take the form of medical foods.

By “medical food” it is meant a food which is formulated to be consumed or administered with or without the supervision of a physician and which is intended for a specific dietary management or condition for which distinctive nutritional requirements, based on recognized scientific principles, are established by medical evaluation.

Various aspects and embodiments of the present invention are defined by the following numbered clauses:

1. A method for increasing video game performance in a subject, comprising: providing the subject with a composition comprising from about 2 mg to about 800 mg of paraxanthine.
2. The method of clause 1, wherein paraxanthine is present in the composition in amount from about 20 mg to about 600 mg.
3. The method of clause 2, wherein paraxanthine is present in the composition in amount from about 50 mg to about 400 mg.
4. The method of any of clauses 1-3, wherein video game performance is assessed by a measure selected from: accuracy, decision making, reaction time, hand-eye coordination, or game performance.
5. The method of any of clause 5, game performance in the subject is increased from about 10% to about 70% following administration of the composition.
6. The method of any of clauses 1-5, wherein the composition further comprises one or more additional active agents.
7. The method of clause 6, wherein in the one or more active agents are selected from: omega-3 fatty acids, vitamin D, vitamin B, protein, selenium, fast digestive carbohydrates, vitamin K, calcium, vitamin A, ashwagandha (Withania somnifera), Acetyl L-Carnitine, tyrosine, N-acetyl-L-tyrosine, Ergothioneine, tryptophan, 5-HTP, arginine, citrulline, norvaline, GABA, Dopa (Velvet Bean), Kanna (serotonin), L-theanine, phosphatidylcholine, alpha-GPC (L-alpha glycerylphosphorylcholine), Citicoline (Cytidine diphosphate choline (CPD Choline)), Choline Bitartrate, Bacopa Monnieri, Phosphatidylserine, pilocarpine, and cevimeline Amburana cearensis, Lippia sidoides, Paullinia cupana, Plathymiscium floribundum, curcuminoids, caffeine, creatine, green coffee berry, lions mane, niacin, kanna extract, saffron, SAMe, acetyl L-carnitine, huperzine A, lutein/zeaxanthin, rhodiola, theacrine, methylcobalamin, macuna, methyliberine, Solanum asperum, taurine, electrolytes like sodium, potassium, magnesium, and calcium, inositol enhanced arginine silicate, nitrates, adenosine triphosphate, valerian root, quercetin, hesperidin, milk thistle and Solanum asperum.
8. The method of any preceding clause, wherein the composition is administered prior to the onset of play.
9. The method of any preceding clause, wherein the composition is administered during video game play.
10. The method of any preceding clause, wherein the composition is administered at repeated intervals during video game play.
11. The method of any preceding clause, wherein the composition is administered to the subject daily for a period of between about 1 and about 9 weeks.
12. The method of clause 11, wherein the composition administered to the subject daily for at least about 3 weeks.
13. The method of clause 12, wherein the composition administered to the subject daily for at least about 6 weeks.
14. The method of any preceding clause, wherein the paraxanthine is synthetic.
15. The method of any preceding clause, wherein the paraxanthine is derived from a natural source.
16. The method of any preceding clause wherein the composition is a dietary supplement.
17. The method of clause 16, wherein the dietary supplement is powder or a capsule.
18. The method of any preceding clause wherein the composition, wherein the composition is a functional food.
19. The method of clause 18, wherein the functional food is a beverage, nutrition bar, yoghurt, or cereal.
20. A method for enhancing electronic gameplay endurance in a subject comprising: providing the subject with a composition comprising from about 2 mg to about 800 mg of paraxanthine.
21. The method of clause 20, wherein the wherein paraxanthine is present in the composition in amount from about 50 mg to about 400 mg.
22. The method of clause 20, wherein the subject has engaged in electronic game play for at least about 100 minutes.
23. A method for enhancing mood of a subject during electronic gameplay or preventing decrease in mood of a subject during electronic game play comprising administering to the subject a composition comprising from about 2 mg to about 800 mg of paraxanthine.
24. The method of clause 23, wherein administration of the composition to the subject decreases, or prevents the increase of, one or more of tension, depression, anger, fatigue and/or confusion in the subject during electronic game play relative to a subject who has not been administered the composition.
25. The method of clause 24, wherein administration of the composition to the subject decreases, or prevents the increase of, tension and/or anxiety in the subject during electronic game play relative to a subject who has not been administered the composition
26. The method of clause 23, wherein administration of the composition to the subject increases, or prevents the decrease of, vigor in the subject during electronic game play relative to a subject who has not been administered the composition.
27. A nutritional supplement to increasing video game performance in a subject comprising from about 2 mg to about 800 mg paraxanthine.
28. The nutritional supplement of clause 27 wherein the paraxanthine is present in amount from about 20 mg to about 600 mg.
29. The nutritional supplement of clause 27 wherein the paraxanthine is present in amount from about 50 mg to about 400 mg.
30. The nutritional supplement of any of clauses 27-29, wherein the nutritional supplement is a dietary supplement.
31. The nutritional supplement of clause 30, wherein the dietary supplement is powder or a capsule.
32. The nutritional supplement any of clauses 27-29, wherein the nutritional supplement is a functional food.
33. The nutritional supplement of clause 32, wherein the functional food is a beverage, nutrition bar, yoghurt, or cereal.
34. The nutritional supplement any of clauses 27-29, further comprises one or more compounds selected from the list consisting of: omega-3 fatty acids, vitamin D, vitamin B, protein, selenium, fast digestive carbohydrates, vitamin K, calcium, vitamin A, ashwagandha (Withania somnifera), Acetyl L-Carnitine, tyrosine, N-acetyl-L-tyrosine, Ergothioneine, tryptophan, 5-HTP, arginine, citrulline, norvaline, GABA, Dopa (Velvet Bean), Kanna (serotonin), L-theanine, phosphatidylcholine, alpha-GPC (L-alpha glycerylphosphorylcholine), Citicoline (Cytidine diphosphate choline (CPD Choline)), Choline Bitartrate, Bacopa Monnieri, Phosphatidylserine, pilocarpine, and cevimeline Amburana cearensis, Lippia sidoides, Paullinia cupana, Plathymiscium floribundum, curcuminoids, caffeine, creatine, green coffee berry, lions mane, niacin, kanna extract, saffron, SAMe, acetyl L-carnitine, huperzine A, lutein/zeaxanthin, rhodiola, theacrine, methylcobalamin, macuna, methyliberine, taurine, electrolytes like sodium, potassium, magnesium, and calcium, inositol enhanced arginine silicate, nitrates, adenosine triphosphate, valerian root, quercetin, hesperidin, milk thistle, and Solanum asperum.
35. The nutritional supplement of any of clauses 27-34, wherein the paraxanthine is derived from a natural source.
36. The nutritional supplement of any of clauses 27-34, wherein the paraxanthine is synthetic.

While multiple embodiments are disclosed, still other embodiments of the disclosure will become apparent to those skilled in the art from the following detailed description, which shows and describes illustrative embodiments of the disclosed compositions, systems and methods. As will be realized, the disclosed compositions, systems and methods are capable of modifications in various obvious aspects, all without departing from the spirit and scope of the disclosure. Accordingly, the drawings and detailed description are to be regarded as illustrative in nature and not restrictive.

### EXAMPLES

The following examples are put forth so as to provide those of ordinary skill in the art with a complete disclosure and description of certain examples of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

### Example 1

### Methods

In a randomized, double-blind, placebo-controlled, parallel group study, 9 healthy men and women, 18-40 years of age, with self-reported history of playing video games for 5 or more hours per week for 6 months prior to screening and a Body Mass Index between 18.5-24.9 kg/m2. The subjects consumed 200 mg (n=5) or a matching placebo (n=4) for 6 weeks, and their first and last dose was consumed inside the laboratory in front of study personnel. Subjects were asked to not consume any caffeine or alcohol for 12 hours before study visit. The effectiveness of study-products (paraxanthine and placebo) was assessed by measuring in-game performance, cognition during gaming (Stroop Test) and mood status during gaming (Profile of Mood States (POMS)).

### Stroop Color and Word Test

The Stroop Color and Word Test (SCWT) was administered after 0, 3, and 6 six weeks of supplementation to assess cognitive performance during gaming throughout the study protocol. The Stroop Test was completed pre-dose, after 60 minutes of gaming and immediately after completing 120 minutes of video gaming during all study visits. The SCWT is a neuropsychological test that follows the procedures of Stroop (Stroop 1935). Briefly, the SCWT (with congruency) was administered for five continuous minutes via an online software application. One of four words (red, yellow, green, blue) appeared at random, until a selection was made, then a new word appeared at random. Subjects selected the response which matches the color of the ink of the randomly selected word (not the color that the word described). The number of correct answers served as the basis of a total Stroop score.

### Profile of Mood States (POMS)

The POMS was completed pre-dose, after 60 minutes of gaming and immediately after completing 120 minutes of video gaming during all study visits. The POMS measured six subscales of mood, including tension-anxiety, depression-dejection, anger-hostility, vigor-activity, fatigue-inertia, and confusion-bewilderment. Subjects were asked to use a 5-point Likert scale (0=not at all, 1=a little, 2=moderately, 3=quite a bit, 4=extremely), to review a list of 65 words (feelings) and rank the degree to which they are experiencing that feeling at the moment. Since high vigor-activity scores reflect good moods or emotions, whereas low scores in the other subscales reflect a good mood or emotion, the total mood disturbance (TMD) score was computed by adding the five negative subscale scores (tension-anxiety, depression-dejection, anger-hostility, fatigue-inertia, and confusion-bewilderment) and subtracting the vigor-activity score. Higher numeric values of the TMD score indicate a greater degree of mood disturbance.

### Video Game Performance

All study participants played the game Super Smash Brothers Ultimate (Nintendo, Release date: Dec. 7, 2018). Each study participant arrived, completed all necessary testing and began playing the game. Game consoles and controllers were available, but if participants wanted to bring their own controller, they were allowed to do so. The game was be set to the same level and strategy for each player. During each session, study participants played the game for 60 minutes and took a break to complete the Stroop Test and POMS. Participants then played the game for an additional 60 minutes before completing their third tests. The following variables were recorded after each hour of game play and were analyzed to assess the impact of supplementation on gaming performance:

### Primary Performance Outcomes

Falls (own deaths)

Damage Taken

Hit Percentage

### Results

### Stroop Color and Word Test

Gaming reduced Stroop test scores by 8% after 1 hour and by 37% after 2 hours. 3 weeks of paraxanthine ingestion increased cognitive performance by 25% pre-gaming, by 39% after one hour of gaming, and by 89% after two hours of gaming compared to baseline. Continued ingestion of paraxanthine for 6 weeks increased cognitive performance by 25% pre-gaming, by 89% after one hour of gaming, and by 119% after two hours of gaming compared to baseline. No habituation was observed with paraxanthine ingestion, even after 6 weeks. After 6 weeks, gaming for 1 and 2 hours did increase cognition, which is in contrast to pre-supplementation cognitive performance, which declined the longer the subjects played.

### POMS

Paraxanthine ingestion improved total mood disturbance after 3 and 6 weeks of supplementation, compared to baseline, and compared to placebo. After 3 and 6 weeks of supplementation, total mood disturbance, worsened during continued gaming in the placebo group, an effect that was not observed in the paraxanthine group.

| | | | Placebo | Paraxanthine |
|---|---|---|---|---|
| | | | (PLA, n = 4) | (PX, n = 5) |
| | Visit | Time | Mean ± SD | Mean ± SD |
| Total Mood | Baseline | 0 h | 14.5 ± 15.6 | −2.2 ± 10.5 |
| Disturbance | | 1 h | 9.8 ± 17.7 | −2.6 ± 18.6 |
| (Tension + | | 2 h | 9 ± 16.3 | −0.4 ± 16.1 |
| Depression + | Mean | | 11.1 | −1.7 |
| Anger + Fatigue + | 3 weeks | 0 h | 7.8 ± 21.3 | −6.2 ± 16.6 |
| Confusion − Vigor) | | 1 h | 8.0 ± 13.0 | −6.8 ± 13.3 |
| | | 2 h | 14.0 ± 25.7 | −6.4 ± 15.8 |
| | Mean | | 9.9 | −6.5 |
| | Δ PLA | | | −166% |
| | Δ Baseline | | | −282% |
| | 6 weeks | 0 h | 7.5 ± 22.7 | −8.2 ± 13.8 |
| | | 1 h | 10.5 ± 26.4 | −11.2 ± 12.4 |
| | | 2 h | 13.8 ± 22.3 | −8.8 ± 13.4 |
| | Mean | | 10.6 | −9.4 |
| | Δ PLA | | | −189% |
| | Δ Baseline | | | −453% |

Feelings of tension-anxiety decreased in the paraxanthine group by 51.7% compared to placebo, and by 39.1% compared to baseline after 3 weeks, and by 61.0% compared to placebo and by 50% compared to baseline after 6 weeks. While tension increased in the placebo group the longer the subjects played, tension decreased in the paraxanthine group the longer the subject played. Increased tension and anxiety negatively impacts gaming performance and cognition.

| | | | Placebo | Paraxanthine |
|---|---|---|---|---|
| | | | (PLA, n = 4) | (PX, n = 5) |
| | Visit | Time | mean ± SD | mean ± SD |
| Tension | Baseline | 0 h | 6.5 ± 2.5 | 5.4 ± 2.2 |
| | | 1 h | 4.8 ± 1.7 | 5.6 ± 5.4 |
| | | 2 h | 4.5 ± 2.4 | 2.8 ± 1.3 |
| | Mean | | 5.3 | 4.6 |
| | 3 weeks | 0 h | 4.5 ± 2.4 | 4.0 ± 2.8 |
| | | 1 h | 6.5 ± 4.4 | 2.0 ± 1.2 |
| | | 2 h | 6.5 ± 4.2 | 2.4 ± 1.3 |
| | Mean | | 5.8 | 2.8 |
| | Δ PLA | | +9.4% | −51.7% |
| | Δ Baseline | | | −39.1% |
| | 6 weeks | 0 h | 4.5 ± 2.9 | 2.6 ± 1.8 |
| | | 1 h | 6.3 ± 4.6 | 2.0 ± 1.0 |
| | | 2 h | 7 ± 5.2 | 2.2 ± 1.3 |
| | Mean | | 5.9 | 2.3 |
| | Δ PLA | | +11.3% | −61.0% |
| | Δ Baseline | | | −50.0% |

Vigor increased in the paraxanthine group by 28.9% compared to placebo, and by 10.2% compared to baseline after 3 weeks, and by 59.3% compared to placebo and by 8.4% compared to baseline after 6 weeks.

| | | | Placebo | Paraxanthine |
|---|---|---|---|---|
| | | | (PLA, n = 4) | (PX, n = 5) |
| | Visit | Time | mean ± SD | mean ± SD |
| Vigor | Baseline | 0 h | 16.5 ± 4.2 | 19.2 ± 5.3 |
| | | 1 h | 15.5 ± 6.4 | 17.4 ± 6.5 |
| | | 2 h | 13.0 ± 5.4 | 13.2 ± 6.4 |
| | Mean | | 15.0 | 16.6 |
| | 3 weeks | 0 h | 15.3 ± 4.1 | 20.4 ± 6.8 |
| | | 1 h | 14.5 ± 6.0 | 18.0 ± 10.0 |
| | | 2 h | 12.8 ± 4.6 | 16.4 ± 10.0 |
| | Mean | | 14.2 | 18.3 |
| | Δ PLA | | | +28.9% |
| | Δ Baseline | | | +10.2% |
| | 6 weeks | 0 h | 13.0 ± 5.2 | 18.6 ± 8.6 |
| | | 1 h | 10.8 ± 6.9 | 18.2 ± 9.2 |
| | | 2 h | 10.0 ± 6.3 | 17.2 ± 9.8 |
| | Mean | | 11.3 | 18.0 |
| | Δ PLA | | | +59.3% |
| | Δ Baseline | | | +8.4% |

### Video Game Performance

Paraxanthine supplementation improved in-game performance as measured by the number of deaths. After 3 weeks, own deaths were reduced by 21.7% compared to placebo, and by 15.5% compared to baseline (prior to supplementation). Paraxanthine is especially effective during later stages of gaming (34% improvement over placebo at gaming between 100 and 120 minutes of play). Similar differences from placebo and baseline were observed after 6 weeks of continued paraxanthine supplementation.

| | | | Placebo | Paraxanthine |
|---|---|---|---|---|
| | | | (PLA, n = 4) | (PX, n = 5) |
| | Visit | Time | Mean ± SD | Mean ± SD |
| Falls (Deaths) | Baseline | 20 min | 8 ± 2.83 | 7 ± 3.54 |
| | | 40 min | 12 ± 4.83 | 10.5 ± 5.26 |
| | | 60 min | 10.25 ± 2.06 | 8 ± 3.39 |
| | | 80 min | 13.5 ± 4.8 | 10.8 ± 4.97 |
| | | 100 min | 15.5 ± 1.29 | 11 ± 3.08 |
| | | 120 min | 18.5 ± 4.44 | 16.6 ± 8.59 |
| | Mean | | 13.0 | 10.65 |
| | 3 weeks | 20 min | 5.25 ± 3.2 | 5.75 ± 2.63 |
| | | 40 min | 7.75 ± 3.2 | 8 ± 3.67 |
| | | 60 min | 9.5 ± 4.2 | 7 ± 3.83 |
| | | 80 min | 11.5 ± 3 | 9 ± 3.32 |
| | | 100 min | 15 ± 3.16 | 11.4 ± 4.04 |
| | | 120 min | 19.67 ± 5.51 | 13 ± 3.74 |
| | Mean | | 11.5 | 9.0 |
| | Δ PLA | | | −21.7% |
| | Δ Baseline | | | −15.5% |
| | 6 weeks | 20 min | 7.75 ± 1.26 | 5.2 ± 1.92 |
| | | 40 min | 8.25 ± 4.11 | 8 ± 2.74 |
| | | 60 min | 10.25 ± 3.86 | 7.4 ± 2.7 |
| | | 80 min | 8.5 ± 3.42 | 10 ± 2.55 |
| | | 100 min | 12.5 ± 4.73 | 13.2 ± 2.59 |
| | | 120 min | 22.0 ± 6.38 | 14.2 ± 3.56 |
| | Mean | | 11.5 | 9.7 |
| | Δ PLA | | | −15.6% |
| | Δ Baseline | | | −8.9% |

Paraxanthine supplementation improved in-game performance as measured by damage taken. After 3 weeks, own damage taken was reduced by 9.5% compared to placebo, and by 9.4% compared to baseline (prior to supplementation). Similar differences from placebo (−14.0%) and baseline (−8.4%) were observed after 6 weeks of continued paraxanthine supplementation. Paraxanthine is especially effective during later stages of gaming (30.9% improvement over placebo at gaming between 100 and 120 minutes of play).

| | | | Placebo | Paraxanthine |
|---|---|---|---|---|
| | | | (PLA, n = 4) | (PX, n = 5) |
| | Visit | Time | Mean ± SD | Mean ± SD |
| Damage | Baseline | 20 min | 875.25 ± 183.83 | 981.20 ± 457.06 |
| Taken | | 40 min | 1358.75 ± 115.17 | 1306.75 ± 553.65 |
| | | 60 min | 1313.50 ± 158.10 | 1207.60 ± 425.74 |
| | | 80 min | 1547.75 ± 224.69 | 1527.80 ± 541.49 |
| | | 100 min | 2044.75 ± 23.43 | 1647.20 ± 359.67 |
| | | 120 min | 2352.75 ± 452.18 | 2067.80 ± 803.18 |
| | Mean | | 1,582.1 | 1,460.9 |
| | 3 weeks | 20 min | 758.50 ± 272.75 | 870.00 ± 301.09 |
| | | 40 min | 1056.50 ± 245.71 | 1103.00 ± 253.95 |
| | | 60 min | 1300.25 ± 374.81 | 987.25 ± 396.60 |
| | | 80 min | 1484.25 ± 360.05 | 1351.80 ± 341.08 |
| | | 100 min | 1892.00 ± 322.90 | 1619.60 ± 383.27 |
| | | 120 min | 2275.67 ± 719.11 | 2006.60 ± 376.34 |
| | Mean | | 1,461.2 | 1,323.0 |
| | Δ PLA | | | −9.5% |
| | Δ Baseline | | | −9.4% |
| | 6 weeks | 20 min | 1008.50 ± 100.69 | 723.62 ± 457.07 |
| | | 40 min | 1134.75 ± 357.37 | 1077.40 ± 258.42 |
| | | 60 min | 1286.75 ± 340.14 | 1182.80 ± 317.56 |
| | | 80 min | 1400.75 ± 447.04 | 1425.80 ± 173.33 |
| | | 100 min | 1723.00 ± 469.94 | 1697.60 ± 336.71 |
| | | 120 min | 2782.75 ± 636.63 | 1921.80 ± 379.30 |
| | Mean | | 1,556.1 | 1,338.2 |
| | Δ PLA | | | −14.0% |
| | Δ Baseline | | | −8.4% |

Paraxanthine supplementation improved in-game performance as measured by hit percentage. After 3 weeks, hit percentage increased by 28.5% compared to placebo, and by 6.8% compared to baseline (prior to supplementation). Similar differences from placebo (+16.4%) and baseline (−5.4%) were observed after 6 weeks of continued paraxanthine supplementation.

| | | | Placebo | Paraxanthine |
|---|---|---|---|---|
| | | | (PLA, n = 4) | (PX, n = 5) |
| | Visit | Time | Mean ± SD | Mean ± SD |
| Hit | Baseline | 20 min | 33.5 ± 4.93 | 36.6 ± 11.13 |
| Percentage | | 40 min | 36.25 ± 7.09 | 30.25 ± 11.15 |
| | | 60 min | 38.75 ± 9.18 | 34.6 ± 9.92 |
| | | 80 min | 35.0 ± 7.48 | 34.2 ± 8.23 |
| | | 100 min | 32.0 ± 7.44 | 30.6 ± 5.98 |
| | | 120 min | 22.5 ± 4.8 | 24.4 ± 4.28 |
| | Mean | | 33.0 | 31.7 |
| | 3 weeks | 20 min | 37.25 ± 7.93 | 37.75 ± 9.64 |
| | | 40 min | 35.75 ± 5.12 | 35.8 ± 8.93 |
| | | 60 min | 34.25 ± 3.86 | 38 ± 5.72 |
| | | 80 min | 33.0 ± 6.38 | 35.2 ± 8.17 |
| | | 100 min | 31.75 ± 5.12 | 29.4 ± 8.08 |
| | | 120 min | 20 ± 2.65 | 26.4 ± 3.51 |
| | Mean | | 26.3 | 33.8 |
| | Δ PLA | | | +28.5% |
| | Δ Baseline | | | +6.6% |
| | 6 weeks | 20 min | 28.5 ± 9.88 | 28.09 ± 16.43 |
| | | 40 min | 31.5 ± 8.23 | 42.2 ± 12.46 |
| | | 60 min | 30.5 ± 5.07 | 39.4 ± 6.88 |
| | | 80 min | 29 ± 7.17 | 33 ± 5.43 |
| | | 100 min | 27.75 ± 4.35 | 30.4 ± 4.98 |
| | | 120 min | 24.75 ± 4.43 | 27.2 ± 4.15 |
| | Mean | | 28.7 | 33.4 |
| | Δ PLA | | | +16.4% |
| | Δ Baseline | | | +5.4% |

### Example 2

### Methods

A 52-year-old healthy, male subject consumed either 100 mg of paraxanthine, or 100 mg of caffeine, or a placebo prior to playing the Video game Tetris. Each of the three games were separated by a one-week washout period. In Tetris, the player brings down blocks from the top of the screen. The player can move the blocks around, either left to right and/or you can rotate them. The blocks fall at a certain rate, but the player can make them fall faster if he/she is sure of the positioning. The game Tetris becomes more challenging the longer you play. It becomes faster the better the player performs. The player has to make the same decisions but under a higher level of pressure. Each time the player clears a line in Tetris, the player is awarded more points. Each time the pieces reach the top of the screen, the game is over. Game performance was measured by play time. The player was not allowed to press a button to increase the speed of the current piece's descent or cause the piece to drop and lock into place immediately, known as a “soft drop” and a “hard drop”, respectively.

### Results

Caffeine intake did not increase gaming performance, however, paraxanthine intake increased gaming performance compared to placebo, and compared to caffeine intake. The player reported an increase in anxiety with caffeine supplementation, in contrast to paraxanthine intake, which resulted in an increase gaming performance without increasing perceived feelings of tension and anxiety.

### Example 3

### Methods

A 52-year-old healthy, male subject consumed either 300 mg of paraxanthine, or 300 mg of caffeine, or a placebo in a blinded fashion prior to playing the fixed shooter arcade video game GALAGA. Each game session consisted of 5 different games and the average score over all 5 games was used as the measure of game performance, as well as the high score/personal best from the 5 sessions. Each of the three game sessions were separated by a one-week washout period. In GALAGA, the player mans a lone starfighter at the bottom of the screen, which must prevent the GALAGA forces from destroying all of mankind. The objective of each stage is to defeat all of the GALAGA aliens, which will fly into formation from the top and sides of the screen.

### Results

Average score with placebo for the 5 games was 66,184 with a personal best of 86,310. Paraxanthine increased average game performance to an average of 118,702 (+79.4%) and the personal best to 161,070 (+86.6%). Game performance with caffeine supplementation showed small improvements over placebo and significantly lower scores compared to paraxanthine with average score of 76,110 for the 5 sessions (+15% compared to placebo, and −35.9% compared to paraxanthine) and a personal best of 102,510 (+18.8% compared to placebo, and −36.4% compared to paraxanthine).

What is claimed is:

1. A method for increasing video game performance in a subject, comprising: administering to the subject with a composition comprising from about 2 mg to about 800 mg of paraxanthine.

2. The method of claim 1, wherein paraxanthine is present in the composition in amount from about 20 mg to about 600 mg.

3. The method of claim 2, wherein paraxanthine is present in the composition in amount from about 50 mg to about 400 mg.

4. The method of claim 1, wherein video game performance is assessed by a measure selected from: accuracy, decision making, reaction time, hand-eye coordination, or game performance.

5. The method of claim 1, wherein game performance in the subject is increased from about 10% to about 70% following administration of the composition.

6. The method of claim 1, wherein the composition further comprises one or more additional active agents.

7. The method of claim 6, wherein in the one or more active agents are selected from: omega-3 fatty acids, vitamin D, vitamin B, protein, selenium, fast digestive carbohydrates, vitamin K, calcium, vitamin A, ashwagandha (Withania somnifera), Acetyl L-Carnitine, tyrosine, N-acetyl-L-tyrosine, Ergothioneine, tryptophan, 5-HTP, arginine, citrulline, norvaline, GABA, Dopa (Velvet Bean), Kanna (serotonin), L-theanine, phosphatidylcholine, alpha-GPC (L-alpha glycerylphosphorylcholine), Citicoline (Cytidine diphosphate choline (CPD Choline)), Choline Bitartrate, bacopa Monnieri, Phosphatidylserine, pilocarpine, cevimeline, Amburana cearensis, Lippia sidoides, Paullinia cupana, Plathymiscium floribundum, curcuminoids, caffeine, creatine, green coffee berry, lions mane, niacin, kanna extract, saffron, SAMe, acetyl L-carnitine, huperzine A, lutein/zeaxanthin, rhodiola, theacrine, methylcobalamin, macuna methyliberine taurine, electrolytes, inositol enhanced arginine silicate, nitrates, adenosine triphosphate, valerian root, quercetin, hesperidin, milk thistle, and Solanum asperum.

8. The method of claim 1, wherein the composition is administered prior to the onset of play.

9. The method of claim 1, wherein the composition is administered during video game play.

10. The method of claim 1, wherein the composition is administered at repeated intervals during video game play.

11. The method of claim 1, wherein the composition is administered to the subject daily for a period of between about 1 and about 9 weeks.

12. The method of claim 1, wherein the paraxanthine is synthetic.

13. The method of claim 1, wherein the paraxanthine is derived from a natural source.

14. A method for enhancing electronic gameplay endurance in a subject comprising: providing the subject with a composition comprising from about 2 mg to about 800 mg of paraxanthine.

15. The method of claim 14, wherein paraxanthine is present in the composition in amount from about 50 mg to about 400 mg.

16. The method of claim 14, wherein the subject has engaged in electronic game play for at least about 100 minutes.

17. A method for enhancing mood of a subject during electronic gameplay or preventing decrease in mood of a subject during electronic game play comprising administering to the subject a composition comprising from about 2 mg to about 800 mg of paraxanthine.

18. The method of claim 17, wherein administration of the composition to the subject decreases, or prevents the increase of, one or more of tension, depression, anger, fatigue and/or confusion in the subject during electronic game play relative to a subject who has not been administered the composition.

19. The method of claim 18, wherein administration of the composition to the subject decreases, or prevents the increase of, tension and/or anxiety in the subject during electronic game play relative to a subject who has not been administered the composition

20. The method of claim 17, wherein administration of the composition to the subject increases, or prevents the decrease of, vigor in the subject during electronic game play relative to a subject who has not been administered the composition.
